(19) United States
(12) Patent Application Publication
(10) Pub. No.: US 2009/0163920 A1
(43) Pub. Date: Jun. 25, 2009

(54) FACET FUSION IMPLANT

(76) Inventors: Stephen Hochschuler, Phoenix, AZ (US); Dennis Colleran, North Attleboro, MA (US); Scott Schorer, Duxbury, MA (US); Rob Brown, Lexington, MA (US)

Correspondence Address: CARR LLP (IST), 670 FOUNDERS SQUARE, 900 JACKSON STREET, DALLAS, TX, 75202, US

(21) Appl. No.: 12/168,030

(22) Filed: Jul. 3, 2008

Related U.S. Application Data
(60) Provisional application No. 60/947,746, filed on Jul. 3, 2007.

(51) Int. Cl. A61B 17/58

(52) U.S. Cl. 606/74; 606/324

(57) ABSTRACT

A medical device, method and system are provided for stabilizing and/or for fusing bone material, such as vertebra of the spine. The medical device (fusion device) has a pair of clamp members and an optional anchor member that cooperate to securely attach the fusion device to a facet joint of the spine. The fusion device may slide down a guide wire and driven into facet bone material of a facet joint. The clamp members may be pivoted together to grip adjacent bone facets and promote their fusion. Further features include injecting flowable bone fusion material between adjacent bone facets gripped by the device.

### CROSS-REFERENCE TO RELATED APPLICATIONS

This application relates to, and claims the benefit of the filing date of, co-pending U.S. provisional patent application Ser. No. 60/947,746 entitled FACET FUSION IMPLANT, filed Jul. 3, 2007, the entire contents of which are incorporated herein by reference for all purposes.

### TECHNICAL FIELD

The invention relates in general to skeletal stabilization systems, and in particular to implants, surgical guides, delivery instruments and methods for delivering and attaching implants to bony structures such as a facet of a vertebrae.

### BACKGROUND INFORMATION

The use of posterior rigid fixation devices for the stabilization of the spine has become very popular. Pedicle screw fixation has been the gold standard for posterior stabilization for more than two decades. However, the safety of pedicle screw fixation has come into question. Tissue damage during insertion, screw mal-position, and the potential risk of neurologic and vascular injury have been reported in the literature. The use of a less invasive means of fixation could reduce or prevent the risks involved with pedicle screw fixation.

### SUMMARY

A medical device, method and system are presented for securing such surfaces as bone material of a spine, such as for posterior stabilization or for fusing various segments of the spine. The medical device (facet fusion device) may have a pair of pivotable clamp members and an anchor member that interrelate to securely attach the facet fusion device to, say, a face joint of the spine. Further, the facet fusion device may have a head to help increase force exertion on the bone material. The facet fusion device may have other features which may allow for (flowable) bone fusion material to be injected into the bone.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a front perspective view of one possible embodiment of an facet fusion device;

FIG. 2 is a detailed exploded view of one embodiment of a component which may be incorporated into the facet fusion device of FIG. 1;

FIG. 3 is an exploded view of one possible embodiment of the facet fusion device of FIG. 1;

FIG. 4 is a side cross sectional view of one possible embodiment of the facet fusion device of FIG. 1 in a first position;

FIG. 5 is a side cross sectional view of one possible embodiment of the facet fusion device of FIG. 1 in a second position;

FIG. 6A is a perspective view one possible embodiment of an instrumentation system which may be used with the facet fusion device of FIG. 1;

FIG. 6B is a cross sectional view of the instrumentation system of FIG. 6A engaged to the facet fusion device of FIG. 1;

FIG. 7A is a flow diagram that illustrates one possible method of delivering a facet fusion device;

FIG. 7B is a perspective view of the facet fusion device of FIG. 1 secured to a facet joint; and

FIG. 7C is a posterior view of a segment of the spine with two facet fusion devices of FIG. 1 secured to a pair of facet joints and an interbody spacer implanted between two adjacent vertebrae.

It is important to note the drawings are not intended to represent the only aspect of the invention. Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the invention is intended to encompass within its scope such processes, machines, manufacture, compositions of matter, means, methods, or steps.

### DETAILED DESCRIPTION

Specific examples of components, methods, and arrangements are described below to simplify the present disclosure. These are, of course, merely examples and are not intended to limit the invention from that described in the claims. Well-known elements are presented without detailed description in order not to obscure the present invention in unnecessary detail. For the most part, details unnecessary to obtain a complete understanding of the present invention have been omitted inasmuch as such details are within the skills of persons of ordinary skill in the relevant art.

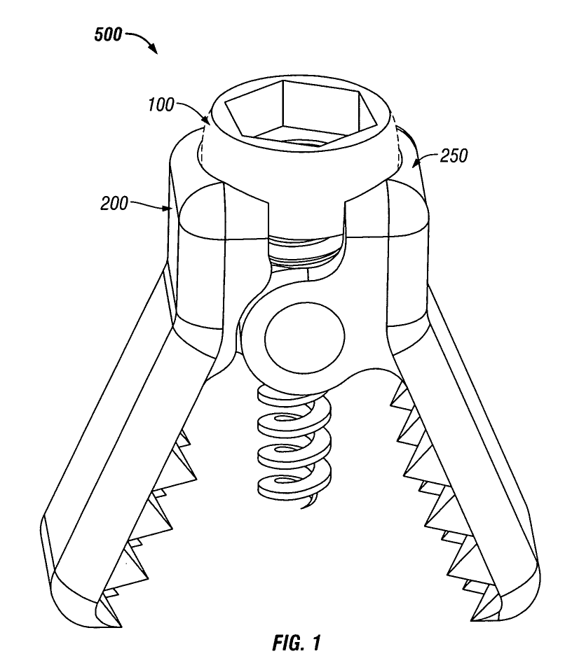

Turning now to FIG. 1 there is presented a front perspective view of one possible embodiment of a facet fusion device 500. The facet fusion device 500 may incorporate a closure member 100 and a pair of clamp members 200 and 250. The facet fusion device 500 may be utilized along with other instruments and implants to stabilize or fuse various segments of the spine. Accordingly, the closure member 100 and the pair of clamp members 200 and 250 may interrelate to securely attach the facet fusion device 500 to a facet joint of a spine to provide for adequate stabilization or fusion.

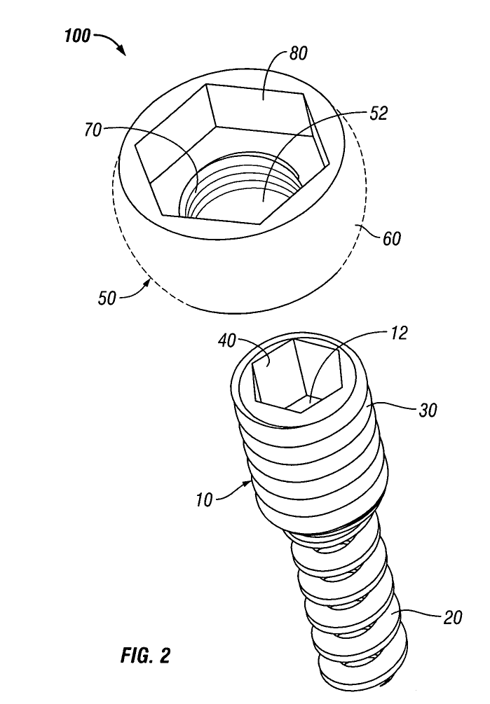

Referring to FIG. 2, there is presented a detailed perspective view of one possible embodiment of the closure member 100. In certain embodiments the closure member 100 may be a one piece integral design or may be assembled from different components, as shown. The closure member 100 may incorporate a fastener 10 and a head 50. The fastener 10 may be coupled to the head 50 prior to or during implantation of the facet fusion device 500 into the spine. The fastener 10 may have an inner surface defining a bore 12 there though that extends along a longitudinal axis of fastener 10. The fastener 10 may have a distal bone anchoring portion 20 at a first end and a coupling element 30 at a second end. The bone anchoring portion 20 may be a threaded fastener (as shown), a tack, a plate, a hook, a staple or other anchoring means known to those skilled in the art. In other embodiments, the fastener 10 may have a corkscrew or spiral type design (as shown in FIG. 1) or may have a traditional thread design. The proximal end of fastener 10 may have an inner surface that defines a non circular recess 40 which may be utilized as a driving means to drive the fastener 10 into or between boney structures. In other embodiments the insertion of fastener 10 may be accomplished with an external driving means. Various geometries may be used for the driving means such as torc, hex, stars, oblong, rectangular, square, and collar shapes.

In certain embodiments, the head 50 may have a spherical outer surface 60 and an inner surface defining a bore 52. In other embodiments the head 50 may include a wedge or a cone design, as will be explained in greater detail below. In certain embodiments, the inner surface of the head 50 may have a distal threaded section 70 to receive the coupling element 30 of the screw 10. The proximal coupling element 30 may have a threaded external surface that engages the threaded section 70 of the head 50. The inner surface of the head 50 may also define non circular recess 80 which may be utilized as a driving means to transfer torque to the head 50 or the screw 10. In other embodiments torque transfer may be accomplished with an external driving means. Various geometries may be used for the driving means such as torc, hex, stars, oblong, rectangular, square, and collar shapes.

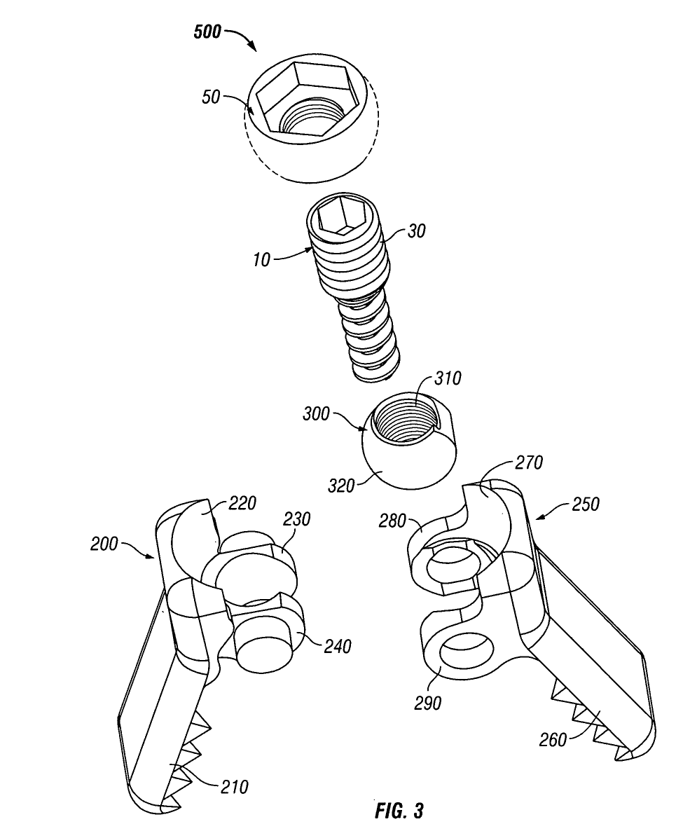

Now turning to FIG. 3 an exploded view of the facet fusion device 500 is shown illustrating the fastener 10, the head 50, the pair of clamp members 200 and 250 and a bushing 300. Each of the pair of clamp members 200 and 250 may have a first end or proximal end and a second end or distal end. The proximal end of each clamp member 200 and 250 may have an engagement surface 220 and 270. In certain embodiments, the distal end may have a clamping portion 210 and 260 with bone gripping surfaces. The bone gripping surfaces may include spikes, textured surfaces or other protrusions which may penetrate and grip or fuse to a boney structure.

In certain embodiments the first clamp member 200 may have a pair of arms 230 and 240 located between the first and second ends that extend out in a first direction and define a channel between the arms. The pair of arms 230 and 240 may have an attachment member or feature, such as a pin or a slot. In certain embodiments the second clamp member 250 may have a pair of arms 280 and 290 located between the first and second ends that extend out in a second direction and defines a channel between the arms. The pair of arms 280 and 290 may each have an attachment member or feature, such as a pin or a slot that mates with a corresponding attachment feature on the pair of arms 230 and 240 of the first clamp member 200 which may allow the clamp members 200 and 250 to pivot in relation to each other. In certain embodiments one pair of arms 280 and 290 (or 230 and 240) may be dimensioned to receive or surround the other pair of arms 230 and 240 (or 280 and 290).

In certain embodiments the facet fusion device 500 may incorporate a bushing 300. The bushing 300 may have an inner surface 310 and outer surface 320. The inner surface 310 may define a bore. The inner surface 310 may be threaded to engage the threaded surface of coupling element 30 of the fastener 10. The outer surface 320 may be at least partially spherical and dimensioned to be received within the channel created by the pair of arms 230 and 240 and/or 280 and 290.

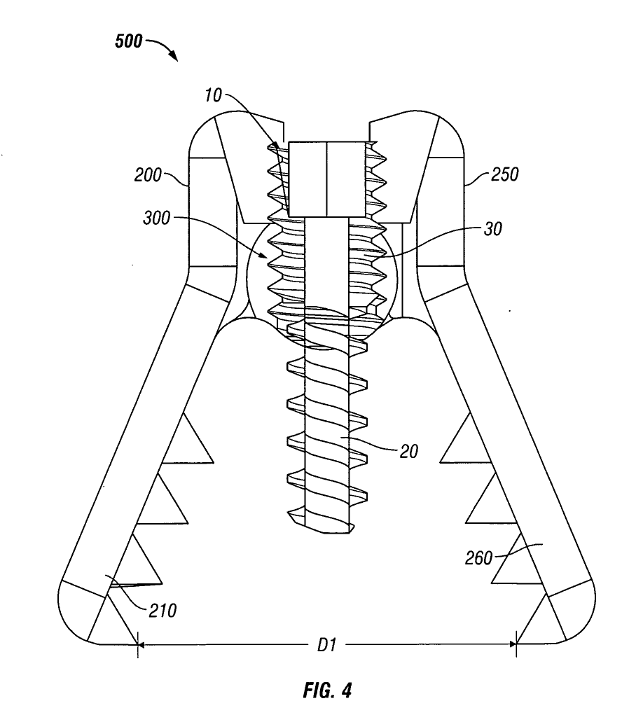

Turning to FIG. 4, a cross sectional view of the facet fusion device 500 is shown illustrating a first position. In certain embodiments, in the first position, the facet fusion device 500 may have a distance “D1” between the distal clamping portions 210 and 260. The clamping members 200 and 250 may attach to a boney structure (not shown), such as with a pair of facets (facet joint). The distance “D1” may be dimensioned to allow the facets to be received between the distal clamping portions 210 and 260.

In certain embodiments, the bushing 300 may be captured between the pair of clamping members 200 and 250. The bone anchoring portion 20 of fastener 10 may pass through the bore of the bushing 300 and may be advanced into or between boney structures (not shown). The bone anchoring portion 20 of the fastener 10 may secure the facet fusion device 500 to the boney structure. As the fastener 10 is advanced into or between the facets (as an example or a boney structure) at least a portion of the coupling element 30 may engage the threaded bore of the bushing 300.

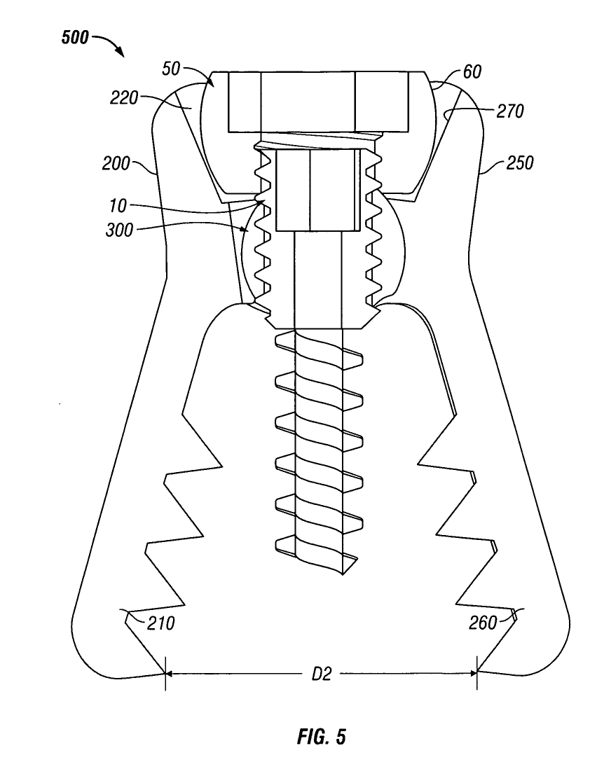

Turning to FIG. 5 a cross sectional view of the facet fusion device 500 is shown illustrating a second position. In the second position the facet fusion device 500 may have a distance “D2” between the distal clamping portions 210 and 260. In certain embodiments the head 50 may threadingly engage the coupling element 30 of the screw 10. As the head 50 is threaded to the coupling element 30 (see, e.g. FIG. 4), the spherical outer surface 60 may exert a force against engagement surfaces 220 and 270 of the clamping members 200 and 250. The force may cause one or both of the clamping members 200 or 250 to pivot in relation to the other clamping member 200 or 250, which may result in the clamping positions 210 and 260 to move to the second position.

In certain embodiments D2 may be less than D1 which may result in the distal clamping portions 210 and 260 clamping or compressing a boney structure (not shown). The compression of bone by the distal clamping portions 210 and 260 may result in creating a localized area of denser bone for improved fixation of fastener 10. In certain embodiments the final implanted facet fusion device 500 may result in multiple points of fixation. For example the screw 10, the first clamping member 200 and the second clamping member 250 may all aid in securing the facet fusion device 500 to a boney structure. In other embodiments the facet fusion device 500 may be used as a clamp without a bone anchoring portion 20 of the fastener 10. In yet other embodiments a fastener 10 may be used without the first clamping member 200 and the second clamping member 250.

In certain embodiments the head 50 may have a first position and a second position. When the head 50 is in the first position there may be little or no force exerted against the engagement surfaces 220 and 270. The head 50 may engage and exert a greater force against the engagement surfaces 220 and 270 in the second position. Other embodiments of force transfer mechanisms, such as the head 50, are also possible which do not utilize a spherical head. The head 50 may be generally cylindrical in shape and may taper to act as a wedge to exert force against the engagement surfaces 220 and 270. The head 50, for example, may have a cam surface that has a first non engagement position and a second engagement position which exerts a force against engagement surfaces 220 and 270. In another embodiment, a wedge member may be inserted between and exerts a force against the two engagement surface 220 and 270. In yet another embodiment a scissor jack or rack and pinion type mechanism may be used to exert a force against engagement surfaces 220 and 270. In still other embodiments, closure type devices may slide over and compress the clamping members 200 and 250 to move clamping portions 210 and 260.

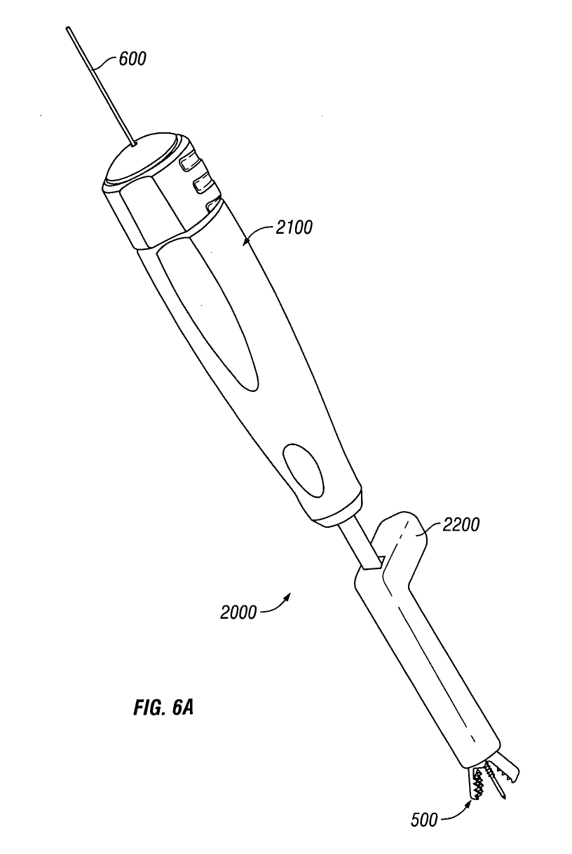

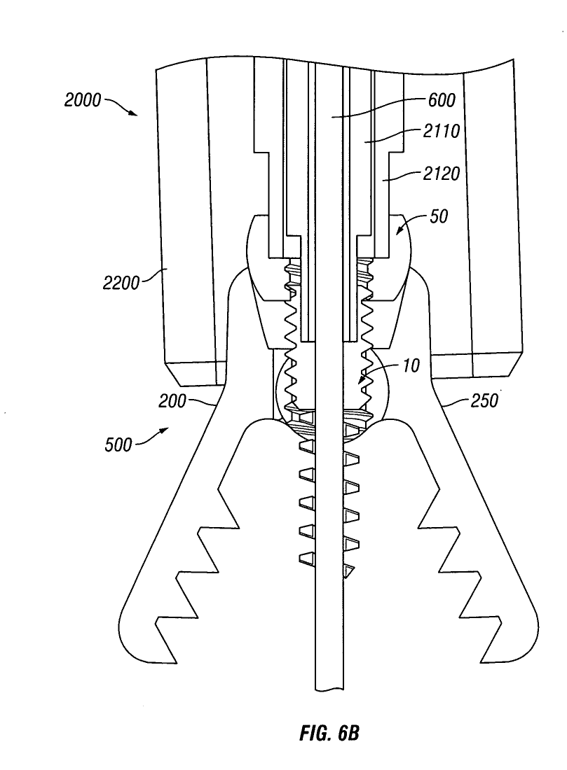

Turning now to FIGS. 6A and 6B one embodiment of a delivery instrument system 2000 is shown which may be used to deliver the facet fusion device 500 to an implantation site. In certain embodiments the delivery instrument system 2000 may include the guide wire 600, a driver 2100 and a counter torque instrument 2200. The driver 2100 may include an inner driver 2110 having a longitudinal bore dimensioned to receive the guide wire 600 and an outer driver 2120 having a longitudinal bore dimensioned to receive the inner driver, as shown in FIG. 6B. The inner driver 2110 may engage the fastener 10 and aid in the insertion of fastener 10 into or between boney structure(s). The outer driver 2120 may engage the head 50 and aid in securing the head 50 to the fastener 10.

The counter torque instrument 2200 may act as a cannula (access device) and/or a counter torque arm. The counter torque instrument 2200 may have a handle and an elongated portion having an inner surface defining a non circular bore (eg rectangular, square or oblong). The facet fusion device 500 may pass down the guide wire 600, through the noncircular bore of the counter torque instrument 2200 and to the implantation site (boney structure). Alternatively, the facet fusion device 500 may slide over the guide wire to the implantation site and then the counter torque instrument 2200 may pass over the guide wire and engage the first end of clamping members 200 and 250. The outer surface of the facet fusion device 500 may correspond to the geometry of the non circular bore of the counter torque instrument 2200 which may act to prevent the facet fusion device from rotating relative to the counter torque instrument 2200. For example, the counter torque instrument 2200 may prevent the facet fusion device 500 from rotating while the fastener 10 and/or the head 50 are inserted and tightened.

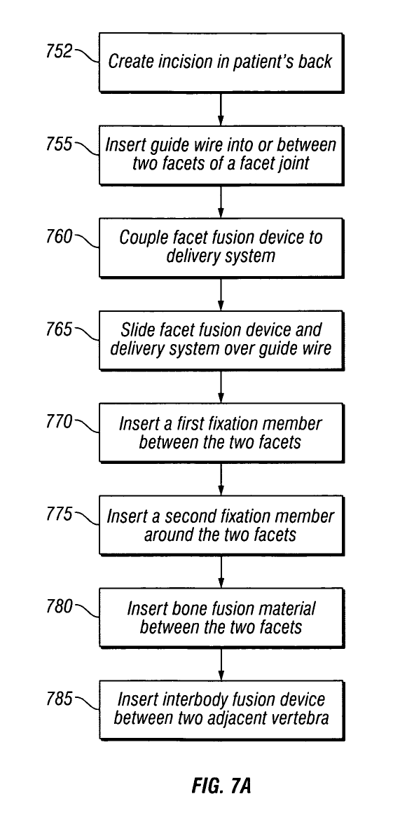

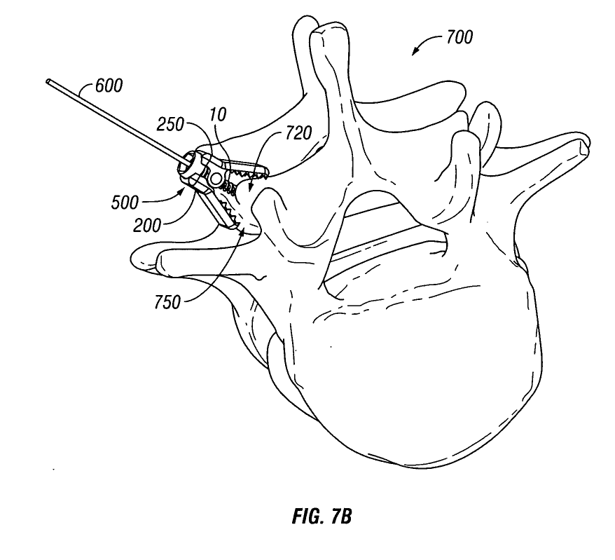

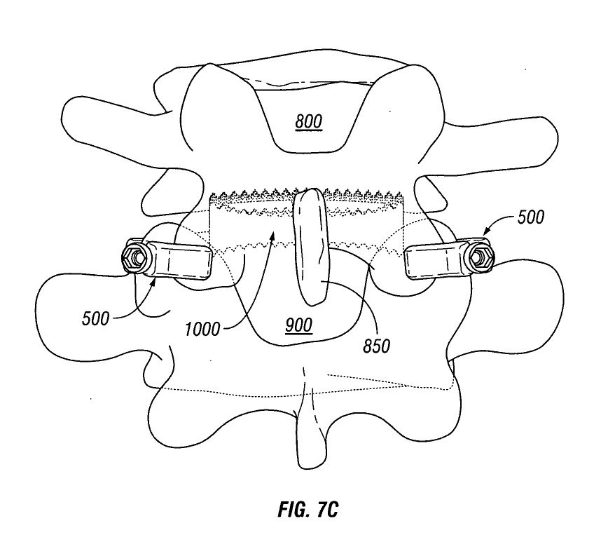

Referring to FIG. 7A, a flow diagram is shown illustrating one possible embodiment of a method for delivering the facet fusion device 500. FIG. 7B, illustrates one embodiment of the facet fusion device 500 delivered and secured to a portion of a spine 700 with the delivery system 2000. A surgeon may start with incision step 752 to aid in access to a pair of facets 720 and 750, of a facet joint. The surgeon may insert a guide wire 600 into a facet or between two facets of a facet joint as shown in step 755 and FIG. 6B. In step 760, the facet fusion device 500 may be delivered to the facet joint with a guide wire 600. In addition to or in lieu of a guide wire 600, a surgeon may use a cannula to deliver the facet fusion device 500. The facet fusion device 500 may slide down the guide wire to the facet joint (or other boney structure) for implantation. The guide wire may pass through the bore 310 of the bushing 300 (see, e.g. FIG. 3) and the bore of the fastener 10. The facet fusion device 500 may be delivered to the facet joint without the head 50 (see FIG. 4) or may be partially coupled to the fastener 10 and later tightened.

In step 760, the driver 2100 and the counter torque instrument 2200 may couple to the facet fusion device 500 (see, e.g., FIG. 6A, 6B), as previously described. The driver 2100, counter torque instrument 2200 and the facet fusion device 500 may slide along the guide wire 600 to a location adjacent to the facets 720 and 750 (e.g. FIG. 7B) as shown in step 765. The facet fusion device 500 may provide for solid fixation of the facets 720 and 750 with an internal and an external fixation means. As previously described, a first fixation member, such as the fastener 10 may be secured between the first facet 720 and the second facet 750 as shown in step 770. The first fixation member (for example, the fastener 10) may exert an outward force on the two facets 720 and 750 pushing them apart from each other. The counter torque instrument 2200 (e.g. FIGS. 6A, 6B) may stabilize the facet fusion device 500 as the fastener 10 is being inserted.

As shown in step 775, a second fixation member (such as the clamping members 200 and 250) may clamp against the first and second facets 720 and 750 (e.g. FIG. 7B). The head 50 (e.g. FIGS. 3, 5) may be inserted and/or tightened to increase the clamp force on the facets 720 and 750. The second fixation member (for example, the clamping members 200 and 250) may exert an inward force against the two facets 720 and 750, which may compress the two facets 720 and 750 toward each other. The counter torque instrument 2200 may stabilize the facet fusion device 500 as the head 50 is inserted and/or tightened.

The securing of the first and second fixation members can be performed in any order. For example, in one embodiment the second fixation member (for example, the clamping members 200 and 250) may be secured first followed by the first fixation member, such as the fastener 10. The clamp members 200 and 250 may exert a compressive force on the facets 720 and 750 which may compact the bone of the facets 720 and 750 to allow for increased fixation of the fastener 10. FIG. 7B shows the facet fusion device 500 implanted, illustrating the fastener 10 secured between the facets 720 and 750 and the clamp members 200 and 250 secured around the facet. After the procedure is completed the guide wire 600 may be removed.

For additional fixation the surgeon in step 780 may insert bone fusion material, such as, BMP (bone morphogenetic protein), autograft or allograft bone ceramic materials, bone cement or other bone ingrowth promoting material between the two facets 720 and 750. In certain embodiments the fastener 10 may be cannulated to allow for a flowable bone fusion material to be injected between the facets 720 and 750. The bone fusion does not necessarily have to be liquid, but may be composed of very small solid pieces. A perforated or corkscrew shaped fastener 10 may allow for improved delivery of bone fusion material. In alternative embodiments the bone fusion material may be a solid material which may be inserted between the two facets 720 and 750 prior to inserting facet fusion device 500.

As shown in step 785 of FIG. 7A, the facet fusion device 500 may be used in conjunction with other fusion devices to give three column support for improved stabilization. An interbody fusion device may be inserted between two adjacent vertebra to aid in fusing the same or adjacent level of the facet fusion device(s) 500. Referring to FIG. 7C, one embodiment of a system for fusion of a pair of adjacent vertebrae 800 and 900 is shown illustrating multiple fusion devices. The facet fusion device 500 is shown implanted to a pair of facet joints on either side of a spinous process 850. An interbody fusion device 1000 may be inserted between two adjacent vertebra to aid in fusing the same or an adjacent level of the facet fusion device(s) 500. An interbody spacer 1000 is shown implanted within the disc space of the pair of adjacent vertebrae 800 and 900. The interbody spacer 1000 may be an ALIF (Anterior Lumbar Interbody Fusion), PLIF (Posterior Lumbar Interbody Fusion), or TLIF (Transforaminal Lumbar Interbody Fusion) spinal fusion device to promote fusion between the adjacent vertebrae 800 and 900. The interbody spacer 1000 may be inserted before or after the insertion of the facet fusion devices 500. This method may result in multiple points of fusion to better stabilize the vertebrae 800 and 900. This method may also result in less surgical time and smaller incisions due to the small size of the facet fusion device 500 and the easy access to the facet joints.

In certain embodiments the facet fusion device 500 may be manufactured using conventional manufacturing techniques such as machining, molding, welding, etc. The facet fusion device 500 may be manufactured from metals (such as stainless steel or titanium), plastics (such as PEEK or UHMWPE) or a combination. In certain embodiments the distal clamping portion may be manufactured from a flexible material to better match the contour of the anatomy being clamped. The delivery instrumentation system 2000 may be manufactured using convention manufacturing techniques such as machining, molding, welding, etc. The delivery instrumentation system 2000 may be manufactured from metals (such as stainless steel or titanium), plastics (such as PEEK or Radel) or a combination.

Although only a few exemplary embodiments of this disclosure have been described in detail above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

What is claimed is:

1. A bone facet fusion device comprising:
an elongated closure member for insertion and securing in alignment with a joint between a pair of adjacent bone facets, the closure member having a proximal end and a distal end;
a clamp secured to the closure member, the clamp comprising first and second clamp members extending outwardly from the proximal end of the closure member and generally toward the distal end of the spacer member; and
an actuation mechanism for moving the first and second clamp members together and towards the closure member to compress a pair of adjacent bone facets against the closure member.

2. The bone facet fusion device of claim 1, wherein the actuation mechanism comprises:
an upper coupling element disposed at the proximal end of the closure member, the coupling element having a threaded outer surface;
a head having an inner surface defining a threaded bore to engage the coupling element of the closure member and engage a first and a second engagement surface of the first clamp member and second clamp member, respectively;
a first arm and a second arm on the first clamp;
wherein the first and second arm extend away from the first clamp member to define a first channel between the first and second arms;
a third and a fourth arm on the second claim member;
wherein the third and fourth arm extend away from the first clamp member to define a second channel between the third and fourth arms;
wherein the first and second arms are coupled to the third and fourth arms such that the first channel and the second channel form a single channel;
wherein the first arm and the second arm each comprise a pin extension, and the third arm and fourth arm each comprise a slot that mates to one of the first and second arm pin extensions, allowing the first clamp and the second clamp to pivot in relation to each other;
a bushing with an inner surface and an outer surface, the inner surface defining a threaded bore for engaging the threaded surface of the coupling element and the outer surface is dimensioned to fit into the single channel between the first arm, the second arm, the third arm and the fourth arm.

3. The bone facet fusion device of claim 1, wherein a lower end of the closure member comprises a bone anchoring portion employable to be driven into bone material, wherein the bone anchoring portion is a corkscrew, a threaded fastener, a tack, a plate, a hook, or a staple.

4. The bone facet fusion device of claim 3, wherein the closure member comprises a bone anchoring portion employable to be driven into bone material, wherein the bone anchoring portion is a corkscrew fastener, a threaded fastener, a tack, a plate, a hook, or a staple; and
the closure member further comprises a head.

5. The bone facet fusion device of claim 4, wherein the head of the closure member comprises a force transfer mechanism for exerting a pivoting torque force on the first and second clamp member.

6. The bone facet fusion device of claim 5, wherein the head of the closure member comprises an inner surface defining a bore; and
wherein the bore of the head comprises a non-circular recess which may receive an external driving means.

7. A method for holding together a plurality of adjacent bone facets, comprising steps of:
inserting a spacer member into a joint between a plurality of adjacent bone facets, the spacer member extending in substantial alignment with surfaces of the plurality of bone facets;
compressing the surfaces of the plurality of bone facets together and against the spacer assembly, in a first position; and
holding the plurality of bone facets and spacer member against relative movement in the first position, such that the surfaces of the plurality of bone facets begin to fuse together.

8. A medical system for placement of a bone fusion device, comprising:
a facet fusion device;
a first clamp;
a second clamp;
means for attaching the first clamp and the second clamp to the facet fusion device;
the first and second clamps comprising gripping surface means for gripping bone material, wherein the gripping surface means are disposed at distal ends of the first clamp and of the second clamp;
means for pivoting the first and second clamps between a first relatively open position and a second relative closed position for gripping bone material between the gripping surface means of the first and second clamps; and
anchor means for inserting into bone material gripped between the gripping surface means and for injecting bone fusion material to the gripped bone material.

9. The medical system for placement of a bone fusion device of claim 8, further comprising delivery means to deliver the facet fusion device to bone material to be gripped, the delivery means further comprising:
a guide wire; and
means for driving the anchor means along the guide wire and into bone material to be gripped.
